(19) United States
(12) Plant Patent Application Publication
(10) Pub. No.: US 2009/0188013 P1
(43) Pub. Date: Jul. 23, 2009

(54) CALIBRACHOA PLANT NAMED 'CALWHIPI'

(50) ;

(75) Inventor: Theodorus Cornelis Maria van Kleinwee, Hoorn (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 12/009,289

(22) Filed: Jan. 17, 2008

(51) Int. Cl. A01H 5/00

(52) U.S. Cl. PLT/413

(57) ABSTRACT

A new Calibrachoa plant named ‘Calwhipi’ particularly distinguished by its high pH tolerance, its rather late flowering, its medium sized downstairs oriented flowers with characteristic color combination of pink and white.

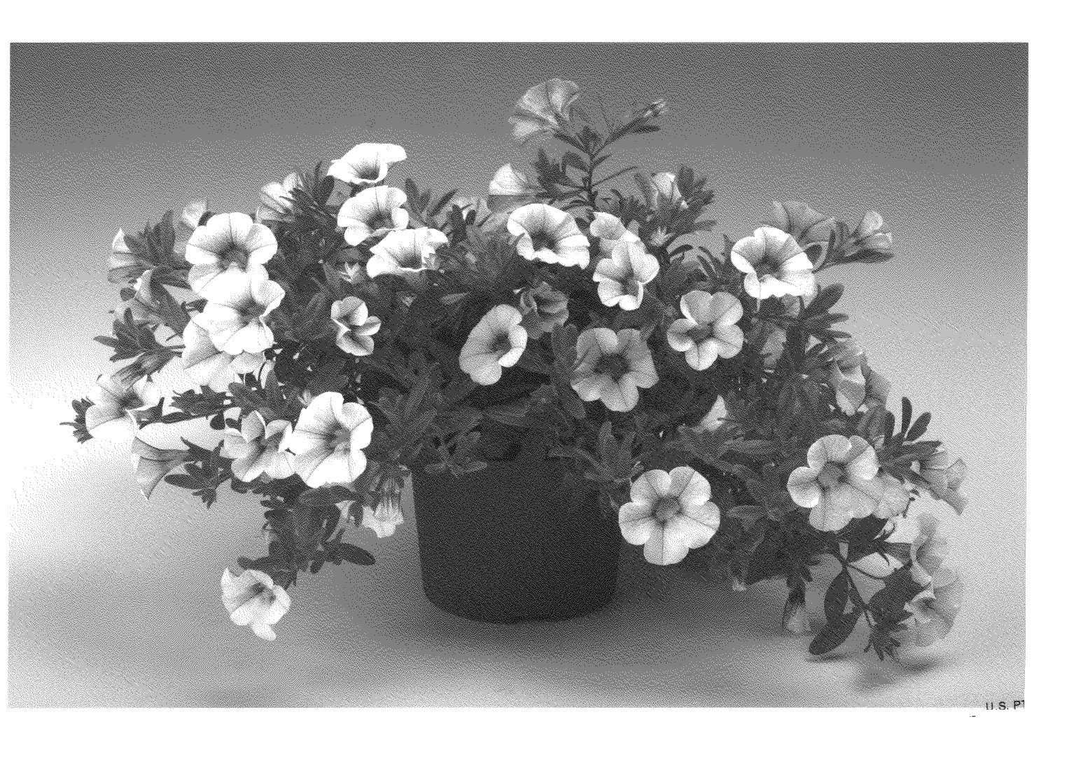

### LATIN NAME OF THE GENUS AND SPECIES OF THE PLANT CLAIMED

Calibrachoa hybrida

### VARIETAL DENOMINATION

‘Calwhipi’

### BACKGROUND OF THE NEW PLANT

The present invention relates to a new and distinct cultivar of Calibrachoa plant, botanically known as Calibrachoa hybrida, and hereinafter referred to by the cultivar name ‘Calwhipi.’

The new Calibrachoa is a product of a planned breeding program conducted in Enkhuizen, Netherlands. The new Calibrachoa cultivar is vigorous with attractive and unique flower coloration.

The new Calibrachoa originates from a cross-pollination in July 2004 of a proprietary selection of Calibrachoa hybrida identified as code number ‘B4208A,’ not patented, as the female, or seed parent, with a proprietary selection of Calibrachoa hybrida identified as code number ‘C4460,’ not patented, as the male, or pollen parent. The new Calibrachoa was selected as a single plant from the resulting progeny of the cross-pollination in a controlled environment in Enkhuizen, Netherlands in May 2005.

Asexual reproduction of the new cultivar by terminal cuttings at Enkhuizen, the Netherlands since August 2005, has shown that the unique features of this new Calibrachoa are stable and reproduced true to type in successive generations.

### BRIEF DESCRIPTION OF THE PHOTOGRAPHS

The accompanying colored photographs illustrate the overall appearance of the new cultivar, showing the colors as true as it is reasonably possible to obtain in colored reproductions of this type. Colors in the photographs may differ slightly from the color values cited in the detailed botanical description which accurately describe the colors of the new Calibrachoa.

### SUMMARY OF THE INVENTION

The following traits have been repeatedly observed and are determined to be the unique characteristics of ‘Calwhipi’. These characteristics in combination distinguish ‘Calwhipi’ as a new and distinct cultivar:
1. High pH tolerance: No yellow leaves at high pH conditions
2. Medium size flowers
3. Rather late flowering
4. Characteristic flower color combination of pink and white.
5. Downstairs oriented flowers

In side-by-side comparisons conducted in Enkhuizen, Netherlands, plants of the new Calibrachoa differed from plants of the female parent selection in the following characteristics:
1. Plants of the new Calibrachoa flower later than plants of the female parent selection.
2. Plants of the new Calibrachoa flower less rich than plants of the female parent selection.

In side-by-side comparisons conducted in Enkhuizen, Netherlands, plants of the new Calibrachoa differed from plants of the male parent selection in the following characteristics:
1. Plants of the new Calibrachoa flower more rich than plants of the male parent selection.
2. Plants of the new Calibrachoa are more branched than plants of the male parent selection.

TABLE 1
DIFFERENCES BETWEEN THE NEW CULTIVAR
‘CALWHIPI’ AND A SIMILAR CULTIVAR

| | | ‘Sunbelkuriho’ (U.S. Plant |
|---|---|---|
| | ‘Calwhipi’ | Pat. No. 17,615) |
| Flower color | White, RH5155C with pink, | White, RHS N155B |
| | RHS N78B | |
| Plant habit | Upright to cascading | Strongly cascading |
| Flower | Big, 2 cm | Smaller, 1.5 cm |
| diameter | | |

### BOTANICAL DESCRIPTION OF THE PLANT

Plants used in the aforementioned photographs and for the following description were grown under conditions which closely approximate commercial production conditions during the spring and summer in a glass-covered greenhouse in Enkhuizen, Netherlands. Plants were grown in containers and were described approximately 20 weeks after planting rooted cuttings. During the production of the plants, the day temperatures ranged from 20° to 30° C., night temperatures ranged from 10° to 15° C.

In the following description, color references are made to The Royal Horticultural Society Colour Chart, 2001 Edition, except where general terms of ordinary dictionary significance are used.
The plant:
   Classification.—Botanical: Calibrachoa hybrida.
Parentage:
   Female parent.—Proprietary selection ‘B4208A,’ not patented.
   Male parent.—Proprietary selection ‘C4460,’ not patented.
Propagation:
   Type.—Terminal cuttings.
   Time to initiate roots.—7 days.
   Time to develop roots.—14 days.
   Root description.—Fine, fibrous.
   Rooting habit.—Freely branching.
Plant description:
   Form.—Annual flowering plant; upright to cascading; uniformly mounded; plants eventually become spherical in overall shape. Freely branching with lateral branches forming at every node. Vigorous growth habit.
   Usage.—Appropriate for hanging baskets, window boxes and patio containers.
   Plant height (from soil level to top of plant plane).—8-10 cm.
   Plant length (from soil level to lateral branches apices).—35-40 cm.
The stem:
   Main branches, length.—About 29 cm.
   Main branches, diameter.—About 6 mm.
   Lateral branches, length.—About 18 mm.
   Lateral branches, diameter.—Up to 3 mm.
   Internode length.—1-1.5 cm.
   Texture.—Pubescent.
   Strength.—Strong, wiry, flexible.
   Color juvenile stem.—152D.
   Color mature stem.—199A.
The foliage:
   Arrangement.—Alternate, simple; sessile.
   Length.—About 22 mm.
   Width.—About 6 mm.
   Shape.—Oblanceolate to elliptic.
   Apex.—Obtuse to slightly acute.
   Base.—Attenuate.
   Margin.—Entire.
   Texture, upper and lower surfaces.—Pubescent.
   Venation.—Pinnate.
   Color.—Developing foliage, upper and lower surfaces: 144A Fully expanded foliage Upper surface: 146A Venation upper surface: 145A Lower surface: 146B Venation lower surface: 145A.
The flower:
   Flower type and habit.—Flowers face upright or outward; single, axillary. Flowers not persistent. Freely flowering with flowers forming at every axil.
   Natural flowering season.—Spring until frost in the autumn; flowering continuous.
   Flower longevity on the plant.—5 days.
   Flower size.—Diameter: About 2 cm Depth (height): About 1.8 cm Tube length: About 1.2 cm Throat diameter, distal end: About 6 mm Tube diameter, proximal end: About 1 mm.
   Flower buds.—Length: About 1.8 cm Diameter: About 4 mm Shape: Oblong Color: Towards base 154C; towards apex 157A.
   Corolla.—Arrangement/appearance: Single whorl of five petals, fused into flared trumpet. Petal length from throat: About 9 mm Petal width: About 11 mm Petal shape: Obdeltoid with a rounded apex Petal margin: Entire Petal texture, upper and lower surfaces: Smooth, glabrous; satiny.
   Color.—When opening, upper surface: Towards center N78B; towards margin 155C; venation 145D When opening, lower surface: 155A; venation 1C Fully opened, upper surface: Towards center N78D; towards margin much whiter than 155C; venation 145D. Fully opened, lower surface: Whiter than 155C; venation 145D Flower throat (inside): 1A; venation 1B Flower tube (outside): 1C.
   Sepals.—Arrangement/appearance: Single whorl of five sepals, star-shaped Length: 9-11 mm Width: About 1.8 mm Shape: Lanceolate Apex: Obtuse Margin: Entire Texture, upper and lower surfaces: Pubescent Color, upper and lower surfaces: 144A.
   Peduncles.—Length: About 11 mm Width: About 8 mm Strength: Flexible, wiry Texture: Densely pubescent Color: 146C.
   Reproductive organs.—Stamens, quantity per flower: 5 Anther shape: Two-parted, rounded, fused at the top Anther length: 0.9 mm Anther diameter: 0.9 mm Anther color: 16B Pollen amount: Moderate Pollen color: 8B Pistils, quantity per flower: 1 Pistil length: About 8 mm Stigma shape: Ovate Stigma color: 144B Style length: About 6 mm Style color: 145A Ovary color: 145A.
Seed development:
   Fruit length.—About 6 mm.
   Fruit width.—About 4 mm.
   Fruit color.—199B.
   Seed length.—About 1 mm.
   Seed width.—About 0.5 mm.
   Seed color.—200B.
   Number of seeds per fruit.—25-30.
Disease resistance: No disease/pest resistance has been observed to date.
Temperature tolerance: Plants of the new Calibrachoa have been observed to be tolerant to temperatures ranging from 4° to 30° C.

What is claimed is:

1. A new and distinct variety of Calibrachoa plant named ‘Calwhipi’, as substantially illustrated and described herein.
